(19) United States
(12) Patent Application Publication
(10) Pub. No.: US 2004/0077985 A1
(43) Pub. Date: Apr. 22, 2004

(54) Method of replenishing cells damaged by treatment for cancer

(76) Inventor: Donnie Rudd, Sugar Land, TX (US)

Correspondence Address: W. Dennis Drehkoff, Ladas & Parry, Suite 1200, 224 South Michigan Avenue, Chicago, IL, 60604, US

(21) Appl. No.: 10/652,376

(22) Filed: Aug. 29, 2003

Related U.S. Application Data
(63) Continuation-in-part of application No. 10/233,344, filed on Sep. 3, 2002.

(51) Int. Cl. A61M 37/00; C12N 1/00; C12M 1/12; A61B 19/00

(52) U.S. Cl. 604/4.01; 604/6.01; 128/898; 435/243; 435/297.1; 435/818

(57) ABSTRACT

Disclosed herein is a method of replenishing cells damaged by treatment for cancer. The method comprises removing blood cells from a primate mammal, controllably expanding the cells at a rate which produces an expansion factor of at least 700% within 7 days while maintaining their three-dimensional geometry and their cell-to-cell support and cell-to-cell geometry, removing any toxic materials from the blood cells, and reintroducing the cells into the primate mammal within a time period sufficient to prevent the primate mammal from suffering decreased mobility due to loss of hematopoietic or other cells.

### BACKGROUND OF THE INVENTION

The present invention relates to a method of replenishing cells damaged by treatment for cancer.

One of the worse side effects of the use of chemotherapy in the treatment for cancer is the loss of energy by the patient due to loss of red blood cells. In the process of destroying cancer cells, chemotherapy often causes damage to other rapidly dividing cells, such as the bone marrow cells. Bone marrow is responsible for producing red blood cells, white blood cells, and platelets. The reduced activity of the bone marrow is named myelosuppression. Chemotherapy and radiation can depress the number of red blood cells to a low level and eventually produce tiredness, lack of energy, and anemia. Myelosuppression is the dose-limiting toxicity of most highly effective chemotherapeutic agents. In recent years this limitation has been overcome through the use of SC transplantation (SCT). In fact, SCT performed after high-dose chemotherapy allows further escalation of dose intensity, thus increasing survival in many patients with advanced malignant diseases. Nevertheless, most patients treated with SCT experience prolonged neutropenia and thrombocytopenia resulting in increased morbidity and mortality.

Labeled by some as cancer's number one side effect, fatigue is part of the illness of 72% to 95% of patients with cancer. Chronic or acute—some describe it as “hitting a wall”—the fatigue experienced by patients with cancer differs from that of healthy people. It is debilitating and depressing, it interferes with normal activities, and it is a barrier to a person's enjoyment of life. The National Cancer Institute describes fatigue's social implications as potentially “profound.”

Fatigue, long discounted, has become more prominent because therapies have become more aggressive and exacerbated it and because health professionals have acknowledged it as a dose-limiting toxicity of therapy and as a quantifiable and treatable side effect. It is emerging as a serious topic of research, which encompasses biochemical, pathophysiologic, psychologic, and behavioral variables. Unfortunately, while medical science has been making steady progress in treating cancer itself, cancer related fatigue is frequently over-looked, under-recognized and under-treated. Aside from the discomfort of feeling exhausted, fatigue can pose a number of obstacles to coping with cancer and reaping the full benefits of available treatments. Fatigue can significantly interfere with a patient's quality of life and may limit the number of chemotherapy cycles that could be administered, which may limit the effectiveness of treatment altogether.

In the past, the preferred treatment for fatigue associated with cancer treatment has been the administration of medication such as epoetin alfa (Procrit), or when the condition becomes severe, a transfusion of red blood cells.

None of the currently available medications, such as epoetin alfa, provide full relief from fatigue due to chemotherapy. While they assist in reducing some of the problems and providing some relief, the medications also have side effects, which create a new series of problems for the patient. Likewise, a transfusion of red blood cells is generally administered only after the patient has suffered the worst effects of the fatigue.

It can therefore be seen that a need exists to minimize the fatigue associated with chemotherapy or radiation for cancer in order to provide a better quality of life for patients undergoing treatment for cancer.

### SUMMARY OF THE INVENTION

The present invention is a method of replenishing cells damaged by treatment for cancer comprising removing blood cells from a primate mammal, controllably expanding the cells a rate which produces an expansion factor of at least seven times within 7 days while maintaining their three dimensional geometry and their cell-to-cell support and cell-to-cell geometry, removing any toxic materials from the blood cells, and reintroducing the cells into the primate mammal within a time period sufficient to prevent the primate mammal from suffering decreased mobility due to loss of hematopoietic or other cells. The damage by treatment for cancer can be from bone marrow transplantation, chemotherapy, or radiation. Preferably, the reintroduction of the cells is within a rather short period of time, preferably within one week after the cancer treatment procedure, but should be no later than within one month after the cancer treatment procedure.

In the present invention, the number of colony-forming units (CFU) granulocyte-macrophage (CFU-GM) and of CFU-granulocyte-macrophage-erythroid-megakaryocyte (CFU-GEMM) will increase about 7-fold and about 9-fold, respectively, by day 7 and the number of burst-forming units-erythroid (BFU-E) will increase about 2.7-fold by day 5 of culture. Significant increases in the numbers of cells expressing CD34+, CD34+/CD38+, CD34+/CD33+, CD34+/CD15+, and CD34+/CD90+and significant declines in the numbers of cells expressing CD34+/CD38- and CD19 surface antigens will occur.

Recombinant hematopoietic growth factors have provided the clinician with a useful tool for treating patients with chemotherapy-induced myelosuppression. These factors include myeloid growth factors such as granulocyte colony-stimulating factor (G-CSF) and granulocyte-macrophage colony-stimulating factor (GM-CSF) which decrease the duration of neutropenia and the incidence of serious infections.

It is an object of this invention to provide a method for replenishing cells damaged by treatment for cancer.

It is a further object of this invention to provide a method for reducing fatigue in patients being treated for cancer with chemotherapy or radiation.

These and still other objects and advantages of the present invention will be apparent from the description of the preferred embodiments that follow.

### DETAILED DESCRIPTION OF THE INVENTION

This invention may be more fully described by the preferred embodiment as hereinafter described.

In the preferred embodiment of this invention, hematopoietic blood cells are removed from a cancer patient prior to chemotherapy treatment. The blood cells are what are currently referred to as pluripotent adult stem cells. The blood cells are placed in a bioreactor, such as that described is U.S. Pat. No. 5,702,941 which is incorporated herein by reference. The bioreactor vessel is rotated at a speed that provides for suspension of the blood cells to maintain their three-dimensional geometry and their cell-to-cell support and geometry. During the time that the cells are in the reactor, they are fed nutrients and toxic materials are removed. The cells are expanded to a volume substantially greater than the original cells. The patient is then administered chemotherapy.

In still another embodiment of this invention, peripheral blood (PB) cells are obtained from normal stem cell (SC) donors. In brief, mononuclear cells (MNCs) are obtained from the first apheresis product collected from SC donors. Prior to apheresis, each donor is treated with G-CSF 6: g/kg every 12 hr over 3 days and then once on day 4. MNCs are collected by subjecting each donor's total blood volume to 3 rounds of continuous-flow leukapheresis through a Cobe Spectra cell separator.

### Operative Method

A) Collection and Maintenace of Cells

Collected MNCs (0.75×10⁶ cells/ml) are suspended in Iscove's modified Dulbecco's medium (IMDM) (GIBCO, Grand Island, N.Y.) supplemented with 20% either fetal calf serum (FCS) (Flow Laboratories, McClean, Va.), 5% human albumin (HA) or 20% human plasma, 100 ng/ml recombinant human G-CSF (Amgen Inc., Thousand Oaks, Calif.), and 100 ng/ml recombinant human stem cell factor (SCF) (Amgen). The culture mix is injected into 300 ml or 500 ml Life Cell nonpyrogenic plastic bags (Baxter, Deerfield, Ill.) and placed in a humidified incubator at 37EC under an atmosphere of 5% CO₂. The culture bags are inspected daily. On days 2, 5, 6, and 7, each culture is mixed, and a sample is aspirated, counted using the trypan-blue exclusion test. If the concentration of cells in a bag exceeds 0.75×10⁶ cells/ml, then IMDM supplemented with either 20% FCS, 5% HA or 20% human plasma, 100 ng/ml G-CSF, and 100 ng/ml SCF is injected into the bag to adjust the cellular concentration to 0.75×10⁶ cells/ml.

B) Analysis of Hematopoietic Colony-Forming Cells Hematopoietic colony-forming cells are assayed using a modification of a previously described assay. In brief, 10⁵ MNCs are cultured in 0.8% methylcellulose with IMDM, 30% FCS, 1.0 U/ml erythropoietin (Amgen), 50 ng/ml recombinant human GM-CSF (Immunex Corp., Seattle, Wash.), and 50 ng/ml SCF (Amgen). One-milliliter aliquots of each culture mixture are then placed in 35-mm Petri dishes (Nunc Inc., Naperville, Ill.) and incubated in duplicate at 37EC in air in a humidified atmosphere of 5% CO₂. All cultures are evaluated after 7 days for the number of burst-forming uniterythroid (BFU-E) colonies (defined asl aggregates of more than 500 hemoglobinized cells or 3 or more erythroid subcolonies), for the number of colony-forming units granulocyte-macrophage (CFU-GM) colonies of granulocytic or monocyte-macrophage cells or both, and for the number of CFU-granulocyte-erythroid-macrophage-megakaryocyte (CFU-GEMM) containing all elements. Individual colonies are plucked from the cultures with a micropipette and analyzed for cellular composition.

C) Analysis of Lymphocytes

Lymphocytes are analyzed by 2-color staining using the following antibody combinations: CD56+/CD16-PE/CD3-FITC, CD3-PE/CD4-FITC, CD3-PE/CD8-FITC, CD19-PE. Controls include IgG1-PE/IgG1-FITC for isotype and CD14-PE/CD45-FITC for gating. Progenitor cells are analyzed by 3-color staining with the fluorochromes PerCP/PE/FITC using the following antibody combinations: CD45/CD90/CD34, CD45/CD34/CD38, CD45/CD34/CD33, and CD45/CD34/CD15. CD45/IgG1/IgG1 is used as a control. In brief, 10⁶ cells from each donor are incubated with 10:1 of antibodies at 2 EC for 15 minutes in the dark and then washed twice in phosphate buffered saline. Then the cells are resuspended, fixed with 1% formaldehyde, and analyzed on a FACScan flow cytometer (Becton-Dickinson) equipped with CELLQuest software (Becton Dickinson). For analyses of lymphocytes, 10,000 cells are acquired from each tube, and then gated on the basis of the forward and right angle light scatter patterns. The cutoff point is visually set at a level above background positivity exhibited by isotype controls. For analyses of progenitor cells, 75,000 cells from each tube is acquired and then sequentially gated.

D) Increase in Hermatopietic Colony-Forming Cells

Incubation of the donors' PB cells in my tissue culture system significantly increases the numbers of hematopoietic colony-forming cells. A constant increase in the numbers of CFU-GM (up to 7-fold) and CFU-GEMM (up to 9-fold) colony-forming cells is observed up to day 7 with no clear plateau.

E) Increase in CD34+Cells

Incubation of MNCs from normal donors in my tissue culture system significantly increases the numbers of CD34+cells. The average number of CD34+cells increased 10-fold by day 6 of culture and plateaus on that same day. The relative number of CD34+cells co-expressing the myeloid-lineage markers CD15 and CD33 increases significantly by days 5 and 6.

Within one week of the chemotherapy treatment, the expanded cells are reintroduced into the body thereby allowing the body to maintain a sufficient level of replenished cells to overcome the fatigue caused by the chemotherapy.

Even though the preferred embodiment of this invention is described above, it will be appreciated by those skilled in the art that other modifications can be made within the scope of this invention.

What is claimed is:

1. A method of replenishing cells damaged by treatment for cancer comprising removing blood cells from a primate mammal, controllably expanding the cells a rate which produces an expansion factor of at least seven times within 7 days while maintaining their three-dimensional geometry and their cell-to-cell support and cell-to-cell geometry, removing any toxic materials from the blood cells, and reintroducing the cells into the primate mammal within a time period sufficient to prevent the primate mammal from suffering decreased mobility due to loss of hematopoietic or other cells.

2. A method as in claim 1 wherein the treatment for cancer is bone marrow transplantation.

3. A method as is claim 1 wherein the treatment for cancer is chemotherapy.

4. A method as in claim 1 wherein the treatment for cancer is radiation.

5. A method as in claim 1 wherein the reintroduction of the expanded blood cells is accomplished within 1 month of the treatment for cancer.

6. A method as in claim 1 wherein the reintroduction of the expanded blood cells is accomplished within 1 week of the treatment for cancer.

7. A method of replenishing cells damaged by treatment for cancer comprising removing peripheal blood cells from a primate mammal, expanding the cells while maintaining their three-dimensional geometry and their cell-to-cell support and cell-to-cell geometry, removing any toxic materials from the peripheal blood cells, and reintroducing the cells into the primate mammal within a time period sufficient to prevent the primate mammal from suffering decreased mobility due to loss of hematopoietic or other cells.

8. A method as in claim 7 wherein the primate mammal is a human.

9. A method as in claim 7 wherein the treatment for cancer is bone marrow transplantation.

10. A method as is claim 7 wherein the treatment for cancer is chemotherapy.

11. A method as in claim 7 wherein the treatment for cancer is radiation.

12. A method as in claim 7 wherein the reintroduction of the expanded blood cells is accomplished within 1 month of the treatment for cancer.

13. A method as in claim 7 wherein the reintroduction of the expanded blood cells is accomplished within 1 week of the treatment for cancer.
